(19) United States
(12) Patent Application Publication
(10) Pub. No.: US 2001/0047106 A1
(43) Pub. Date: Nov. 29, 2001

(54) PROCESS FOR THE ESTERIFICATION OF (METH)ACRYLIC ACID WITH AN ALKANOL

(76) Inventors: HEINRICH AICHINGER, MANNHEIM (DE); MICHAEL FRIED, HEIDELBERG (DE); GERHARD NESTLER, LUDWIGSHAFEN (DE); HOLGER HERBST, FRANKENTHAL (DE)

Correspondence Address: OBLON SPIVAK MCCLELLAND MAIER & NEUSTADT, 1755 JEFFERSON DAVIS HIGHWAY, FOURTH FLOOR, ARLINGTON, VA, 22202

(21) Appl. No.: 09/341,663

(22) PCT Filed: Jan. 8, 1998
(86) PCT No.: PCT/EP98/00068

§ 371 Date: None

(30) Foreign Application Priority Data
Jan. 20, 1997 (DE) 19701737.1

(51) Int. Cl. C07C 69/74; C07C 69/52; C07C 67/30

(52) U.S. Cl. 560/205; 560/212

(57) ABSTRACT

In a process for esterifying (meth)acrylic acid with an alkanol, oxy ester formed as a byproduct of the esterification is recleaved under acid catalysis with addition of monomeric and/or oligomeric (meth)acrylic acid and water.

The present invention relates to a process for esterifying (meth)acrylic acid with an alkanol in the presence of an esterification catalyst, in which unreacted starting compounds and the methacrylic ester to be formed are separated from the reaction mixture by distillation, with an oxy ester-containing bottom product remaining, and either

a) monomeric and/or oligomeric (meth)acrylic acid is added directly to the bottom product and then the oxy esters present in the bottom product are cleaved by the action of elevated temperature in the presence of acid catalysts different from monomeric and oligomeric (meth)acrylic acid, or

b) the oxy esters are first separated off from the bottom product by distillation, the distillate is admixed with monomeric and/or oligomeric (meth)acrylic acid and then the oxy esters present therein are cleaved by the action of elevated temperature in the presence of acid catalysts different from monomeric and oligomeric (meth)acrylic acid.

The term (meth)acrylic acid, as is customary, designates here acrylic acid or methacrylic acid. The designation oligomeric (meth)acrylic acid means the Michael adducts of (meth)acrylic acid with itself and with the resulting secondary products.

Michael adducts of this type may be characterized by the formula

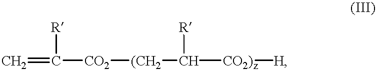

and must be differentiated here from (monomeric) (meth)acrylic acid and from (meth)acrylic acid polymers (which are obtainable by free-radical polymerization of (meth)acrylic acid). It is of importance that the Michael addition of (meth)acrylic acid with itself and with the resulting secondary products is reversible.

Oligomeric (meth)acrylic acid is produced, for example, in the distillation of (for example crude) (meth)acrylic acid (the term “crude” indicates a small amount of aldehyde impurities, in particular, still present) in the bottom phase (cf. DE-A-22 35 326, for example).

Alkyl esters of (meth)acrylic acid, owing to their activated ethylenically unsaturated C═C double bond, are important starting compounds for preparing polymers generated by free-radical polymerization, which are used as glues, for example.

Customarily, alkyl (meth)acrylates are prepared by esterifying (meth)acrylic acid with alkanols at elevated temperature in the liquid phase with or without solvent and in the presence of acid catalysts different from (meth)acrylic acid (cf. DE-A 23 39 519, for example). A disadvantage of this method of preparation is that, under the abovementioned esterification conditions, unreacted starting alcohol forms a compound of the formula I below and unreacted (meth)acrylic acid forms a compound of the formula II, by addition to the ethylenically unsaturated double bond (Michael addition) of alkyl (meth)acrylate already formed, as side reactions.

Successive multiple addition is also possible. In addition, mixed types can occur. These adducts (alkoxy esters and acyloxy esters) are termed oxy esters for short:

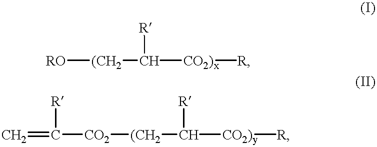

In the preparation of acrylic esters, oxy ester formation is particular problematic, the oxy esters principally formed being alkoxypropionic ester and acyloxypropionic ester where X, Y=1. In the preparation of methacrylic esters, oxy ester formation proceeds to a lesser extent. The formation of oxy esters is described, inter alia, in DE-A 23 39 529. The above laid-open application confirms that the formation of oxy esters is essentially independent of the specific esterification conditions. Oxy ester formation is of particular importance in the preparation of acrylic esters of the C₁C₈-alkanols, in particular the C₄-C₈-alkanols, very particularly in the preparation of n-butyl acrylate and 2-ethylhexyl acrylate.

It is characteristic of the oxy esters that their boiling point is above the boiling points of starting acid, starting alcohol, target ester formed and of any organic solvent used in conjunction.

Any particular esterification reaction mixture is usually worked up in such a manner that unreacted starting compounds and the target ester are separated off from the reaction mixture by distillation, in which case the acid catalyst used for the esterification may be separated off in advance by extracting with water and/or aqueous alkali metal hydroxide solution (cf., for example, Ullmann's Encyclopedia of Industrial Chemistry, Vol. A1, 5th Ed., VCH, pp. 167ff.). The bottom product remaining after such a work-up by distillation contains the oxy esters, which result in a considerable loss in yield.

The use of very varied procedures has therefore been attempted to diminish the problems resulting from the occurrence of the oxy esters.

Thus, JP-A-82/62229 describes the alkaline saponification of the high-boiling esterification residue. Although some of the alcohol and acrylic acid used and β-hydroxypropionic acid are recovered in this manner, it is not possible to recycle them simply and economically to the esterification reaction, owing to their salt content due to the alkaline saponification conditions, which is a disadvantage.

JP-B-72/15936 relates to the reaction of β-alkoxypropionic esters with acrylic acid in the presence of strong acids with the production of acrylic esters (transesterification). However, as a byproduct, an equimolar amount of β-alkoxypropionic acid is produced, which could not be recycled to the (meth)acrylic acid esterification.

JP-A-93/25086 relates to the cleavage of the Michael addition product butyl β-butoxypropionate (see formula I, X=1, R=butyl) at elevated temperature and in the presence of sulfuric acid and an excess of water. It is a disadvantage of this procedure that the conversion rate is only 30%.

JP-A-94/65149 describes the cleavage of Michael addition products I and II in the presence of titanium alkoxides. A disadvantage here is the likewise comparatively low (<60%) conversion rate and the requirement for titanates.

GB-B 923 595 discloses the work-up of the residue of the esterification of acrylic acid with alkanols in the absence of molecular oxygen. It is recommended that, inter alia, all volatile monomers are removed prior to cleavage, the cleavage is carried out in the presence of sulfuric acid and that the cleavage products are removed using an inert gas stream. According to the illustrative examples, the cleavage is carried out at at least 300° C. The residue formed is carbon, which must be mechanically removed from the reactor. Therefore, this procedure is neither economical nor performable on an industrial scale.

CN-A 1,063,678 describes the cleavage of the alkoxypropionic ester present in the esterification residue in the presence of sulfuric acid in a cascade, temperature and catalyst concentration being different in each reactor. A distillation to separate alkanol and acrylate is coupled to the cleavage. The procedure is highly laborious and does not achieve high conversion rates.

CN-A 1,058,390 relates to the cleavage of alkoxypropionic esters in the presence of sulfuric acid, etc., into alkanols and acrylic esters. This procedure is performed in separate steps. Firstly, the cleavage is carried out under reflux, and then the reaction products are distilled off. The acrylic-acid-containing ester residues of the preparation of ethyl acryl/methyl acryl [sick] (ethyl ethoxypropionate, methyl methoxypropionate) are cleaved in the presence of ethanol or methanol, respectively. This procedure also is complicated and does not achieve high yields.

DE-A 19547459 and DE-A 19547485 relate to cleaving the oxy esters in the presence of monomeric or oligomeric (meth)acrylic acid and in the presence of acids different from the abovementioned acids. Formation of the unwanted cleavage byproducts may be markedly decreased by this means, but the reaction rate is unsatisfactory.

U.S. Pat. No. 3 227 746 proposes cleaving alkyl alkoxypropionates in the presence of dehydration catalyst and water. A presence of monomeric or oligomeric (meth)acrylic acid is not incorporated. According to Example 6, butyl butoxypropionate is cleaved in the presence of 100% by weight (based on oxy ester) of 85% strength by weight phosphoric acid and 10% by weight (based on oxy ester) of water. The presence of water, according to U.S. Pat. No. 3,227,746 makes the presence of alkanol superfluous and prevents unreacted alkyl alkoxypropionate from passing over into the distillate. A disadvantage of this procedure is the high amount of catalyst used. In addition, the reaction rate is unsatisfactory, as is the formation of cleavage byproducts.

It is an object of the present invention, therefore, to carry out the cleavage of the oxy esters formed during the esterification of (meth)acrylic acid with an alkanol in a manner more advantageous than in the prior art and to integrate it into the esterification process.

We have found that this object is achieved, accordingly, by a process for esterifying (meth)acrylic acid with an alkanol in the presence of an esterification catalyst, in which unreacted starting compounds and the (meth)acrylic ester to be formed are separated from the reaction mixture by distillation, with an oxy ester-containing bottom product remaining, and either

a) monomeric and/or oligomeric (meth)acrylic acid is added directly to the bottom product and then the oxy esters present in the bottom product are cleaved by the action of elevated temperature in the presence of acid catalysts different from monomeric and oligomeric (meth)acrylic acid or

b) the oxy esters are first separated off from the bottom product by distillation, the distillate is admixed with monomeric and/or oligomeric (meth)acrylic acid and then the oxy esters present therein are cleaved by the action of elevated temperature in the presence of acid catalysts different from monomeric and oligomeric (meth)acrylic acid, which involves

adding additional water for cleavage to the oxy esters separated off by distillation from the bottom product, or adding additional water for cleavage to the bottom product.

Generally, from 5 to 50% by weight, preferably from 10 to 40% by weight, of monomeric and/or oligomeric (meth)acrylic acid is added, based on the oxy esters to be cleaved. Usually, the monomeric and/or oligomeric (meth)acrylic acid is added in a form which is known per se and which is stabilized by polymerization inhibitors. In an expedient manner, the oligomeric (meth)acrylic acid used for the novel process is the bottom phase produced in the purification of crude (meth)acrylic acid by distillation, which bottom phase principally contains compounds of the formula III.

The monomeric (meth)acrylic acid and/or the (meth)acrylic oligomers can be added to the mixture to be cleaved prior to the cleavage. However, they can also be fed separately to the cleavage reactor.

Under the recleavage conditions, the oligomeric (meth)acrylic acids are recleaved, as a result of which free (meth)acrylic acid is continuously produced in the nascent state. In comparison with addition of (meth)acrylic acid in advance, this has the advantage that the added (meth)acrylic acid does not distill off immediately together with the cleavage products, but the cleavage proceeds continuously in the presence of (meth)acrylic acid, which gives rise to a particularly low formation of byproducts (dialkyl ethers, olefins).

The amount of water to be added according to the invention for cleavage, based on the oxy esters to be cleaved, is generally from 0.1 to 20% by weight, preferably from 1 to 10% by weight.

According to an advantageous development of the invention, the process is carried out in the presence of molecular oxygen.

According to a further advantageous development of the invention, in addition to the acid esterification catalyst which may still be present and is different from monomeric and oligomeric (meth)acrylic acid, further acids may be added to the product to be cleaved selected from the group consisting of mineral acids, such as sulfuric acid or phosphoric acid, and organic acids which are different from monomeric and oligomeric (meth)acrylic acid, such as alkylsulfonic or arylsulfonic acids, for example methanesulfonic or p-toluenesulfonic acids.

In this development, the total amount of acid which is different from monomeric and oligomeric (meth)acrylic acid which is then present can be from 1 to 20% by weight, preferably from 5 to 15% by weight, based on the amount of product to be cleaved.

It has proved to be expedient if a stripping gas is passed, as entrainer for the cleavage products, through the product to be cleaved during the process according to the invention, which stripping gas preferably contains molecular oxygen. Expediently, the stripping gas used is air or mixtures of air with inert gas (eg. nitrogen).

The advantages of the process according to the invention are, primarily, that in this process the cleavage firstly proceeds at an increased rate and, secondly, at the same time few byproducts such as ethers or olefins are formed. Thus, inter alia, reduced losses of starting materials, especially alcohols, occur, than with known processes. Furthermore, high rates of cleavage can be achieved and the direct recycling of the cleavage products to the esterification does not give rise to any adverse effect on the (meth)acrylic ester unit.

In the separation of the oxy esters from the bottom product by distillation, the distillation conditions depend on the type of the alcohol component used in the esterification. Generally, from 100 to 300° C. and a pressure of from 1 to 50 mbar are contemplated. Any conventional distillation apparatus is suitable for the distillation process. Since only a simple separation task is to be achieved, a simple splash guard generally suffices, ie. a column is not usually necessary.

For the work-up according to the invention of the oxy esters produced in the bottom product during esterification, or of the oxy ester distillate separated off from the esterification bottom product, a simple heatable stirred reactor having jacketed heating or a heating coil can be used, or else a forced-circulation evaporator, for example a falling-film evaporator or flat evaporator, coupled to a detention vessel, can be used. For improved separation of the cleavage products from the bottom product or oxy ester distillate, a rectification apparatus mounted on the cleavage apparatus, eg. a column containing random or arranged packings or trays, may be expedient. This rectification apparatus is generally operated stabilized with polymerization inhibitors (eg. phenothiazine, hydroquinone monomethyl ether etc.).

Typical conditions for carrying out the process according to the invention of the cleavage of the oxy esters produced in the bottom product during the esterification or separated off from the bottom product are as follows:

| | |
|---|---|
| Catalyst: | at least one acid selected from the |
| | group consisting of mineral acids, such |
| | as sulfuric acid and phosphoric acid, |
| | and organic acids different from |
| | (meth)acrylic acid, such as |
| | alkylsulfonic or arylsulfonic acids, for |
| | example methanesulfonic acid or |
| | p-toluenesulfonic acid |
| Amount of catalyst: | 1-20% by weight, preferably 5-15% by |
| | weight, based on the amount of the |
| | bottom product or on the amount of the |
| | oxy ester distillate separated off from |
| | the bottom product |
| Amount of monomeric and/or | 5-50% by weight, preferably 10-40% by |
| oligomeric (meth)acrylic acid: | weight, based on the amount of the |
| | bottom product, or on the amount of the |
| | oxy ester distillate separated off from |
| | the bottom product |
| Amount of water: | 0.1-20% by weight, preferably 1-10% by |
| | weight, based on the amount of the |
| | bottom product, or on the amount of the |
| | oxy ester distillate separated off from |
| | the bottom product |
| Temperature: | 150-250° C., preferably 180-230° C. |
| Pressure: | preferably atmospheric or reduced |
| | pressure (so that the cleavage products |
| | evaporate immediately) |

Stripping gas, where used: rate: 1-100 1/h × 1

| | |
|---|---|
| Reaction time: | 1-10 hours. |
| Conversion rate: | ≧90% |

The reaction is run, for example, in such a manner that the bottom product to be cleaved is continuously ejected from the workup by distillation of the esterification mixture and is fed to the cleavage reactor together with the cleavage catalyst, the water and the monomeric and/or oligomeric (meth)acrylic acid. However, the reaction can also be carried out discontinuously, ie. batchwise. It is also possible to perform the reaction semicontinuously, in which the product to be cleaved, water and the monomeric and/or oligomeric (meth)acrylic acid are continuously fed to the cleavage reactor which contains the cleavage catalyst, and the bottom product is not removed batchwise from the cleavage reactor until the cleavage is completed. The cleavage products are continuously separated off by distillation and are expediently recycled to the esterification.

If the esterification is carried out in such a manner that the water formed during the esterification is continuously separated off via a rectification column mounted on the esterification reactor, the cleavage products are preferably recycled to the esterification via this rectification column (expediently, recycled material is passed to the lower half of the rectification column).

The monomeric and/or oligomeric (meth)acrylic acid and the water can be fed to the cleavage reactor separately or together, or else in a mixture with the product to be cleaved.

The applicability of the cleavage process described is not restricted to a special nature of the esterification process, as the byproducts of which the oxy esters, that is the addition compounds I and II, arise. Generally the esters are prepared by the customary processes (see Ullmann's Encyclopedia of Industrial Chemistry, Vol. A1, 5th Ed., VCH, pp. 167ff).

A typical example of the conditions under which the esterification preceding the cleavage of the oxy esters can take place may be described in brief as follows:

| | |
|---|---|
| Alcohol: (Meth)acrylic acid | 1:0.7-1.2 (molar) |
| Catalyst: | Sulfuric acid or sulfonic acids |
| | (eg. p-toluenesulfonic acid) |
| Catalyst amount: | 0.1-10% by weight (preferably |
| | 0.5-5% by weight) based on starting |
| | material |
| Stabilization: | 200-2000 ppm of phenothiazine |
| | (based on the weight of the |
| | starting materials) |
| Reaction temperature: | 80-160° C., preferably 90-130° C. |
| Reaction time: | 1-10 hours, preferably 1-6 hours. |

An entrainer (eg. cyclohexane or toluene) may be used to remove the water of esterification. The esterification can be carried out at atmospheric pressure, under reduced pressure or at superatmospheric pressure, either continuously or batchwise.

In the acid-catalyzed esterification of acrylic acid with alkanols, the bottom product resulting after separating off the acid esterification catalyst, the unreacted starting materials and the acrylic ester generally has the following composition:

1-20% by weight of acrylic ester

50-80% by weight of alkoxypropionate (see formula I)

5-30% by weight of acyloxypropionate (see formula II)

Remainder: principally stabilizers (phenothiazine) and polymers.

Further details and advantages of the process according to the invention can be taken from the illustrative example described below.

First of all, a result achieved with a process not according to the invention is to be described with reference to a comparative example.

### Comparative Example 1

A glass circulation reactor (volume: 1 l), heated by a heating tube, was charged with 500 g of an oxy ester distillate, produced from n-butyl acrylate preparation esterification residue freed from the acid esterification catalyst, and 40 g of p-toluenesulfonic acid. The oxy ester distillate contained

11.0% by weight of butyl acrylate,

64.8% by weight of butoxy ester I (R═C₄H₉)

20.5% by weight of acyloxy ester II (R═C₄H₉).

10 l of air were introduced into the mixture per hour.

The cleavage temperature was 195° C. and the operating pressure was 1 atm.

The esterification residue to be cleaved was continuously fed during the cleavage to the cleavage reactor under level control.

The cleavage products were removed in the vapor state and condensed at the top of the column (50 cm×2.8 cm, empty) mounted on the cleavage reactor. In the course of 119.5 hours, 7401 g of mixture (62 g/h) were fed to the cleavage and 7080 g of cleavage products were condensed.

According to gas-chromatographic analysis, the condensate contained:

72.0% by weight of butyl acrylate

13.9% by weight of butanol

4.8% by weight of acrylic acid

1.4% by weight of dibutyl ether

6.6% by weight of butenes

0.2% by weight of butyl butoxypropionate

Conversion rate: 96% by weight, based on oxy ester

### EXAMPLE

### Example of the Procedure According to the Invention

A glass circulation reactor (volume 1 l), heated with a heating tube, was charged with 500 g of the oxy ester distillate from Comparative Example 1, 40 g of p-toluenesulfonic acid, 100 g of acrylic acid (stabilized with 300 ppm of phenothiazine) and 20 g of water. 10 l of air were introduced per hour into the mixture.

The cleavage temperature was 195° C., the operating pressure was 1 atm.

Under level control, the oxy ester distillate to be cleaved, 20% by weight of acrylic acid and water (4% by weight, based on oxy ester distillate) were continuously fed to the cleavage reactor. The cleavage products were condensed at the top of the column (50 cm×2.8 cm, empty) mounted on the reactor.

In the course of 100 hours, 15,250 g of oxy ester distillate (153 g/h), 3050 g of stabilized acrylic acid and 600 g of water were fed to the cleavage and 18,350 g of product mixture were condensed. According to gas-chromatographic analysis, the condensate contained, without the water:

73.8% by weight of butyl acrylate

6.5% by weight of butanol

12.9% by weight of acrylic acid

0.7% by weight of dibutyl ether

2.8% by weight of butenes

<1% by weight of butyl butoxypropionate

Conversion rate: 97% by weight, based on oxy ester.

### Comparison Example 2

The procedure of the example was followed, but without addition of water. Throughput: 108 g/h at 96% by weight conversion rate (based on oxy ester). Byproducts (total of olefin and dibutyl ether): 4.0% by weight, based on condensate.

We claim:

1. A process for esterifying (meth)acrylic acid with an alkanol in the presence of an esterification catalyst, in which unreacted starting compounds and the (meth)acrylic ester to be formed are separated from the reaction mixture by distillation, with an oxy ester-containing bottom product remaining, and either
a) monomeric and/or oligomeric (meth)acrylic acid is added directly to the bottom product and then the oxy esters present in the bottom product are cleaved by the action of elevated temperature in the presence of acid catalysts different from monomeric and oligomeric (meth)acrylic acid or
b) the oxy esters are first separated off from the bottom product by distillation, the distillate is admixed with monomeric and/or oligomeric (meth)acrylic acid and then the oxy esters present are cleaved by the action of elevated temperature in the presence of acid catalysts different from monomeric and oligomeric (meth)acrylic acid,
which comprises adding additional water for cleavage to the oxy esters separated off by distillation from the bottom product, or adding additional water for cleavage to the bottom product.

2. A process as claimed in claim 1, wherein the amount of monomeric and/or oligomeric (meth)acrylic acid added to the product to be cleaved is from 5 to 50% by weight, based on the product to be cleaved.

3. A process as claimed in claim 1, wherein the amount of monomeric and/or oligomeric (meth)acrylic acid added to the product to be cleaved is from 10 to 40% by weight, based on the product to be cleaved.

4. A process as claimed in one of claims 1 to 3, wherein the amount of water added to the product to be cleaved is from 0.1 to 20% by weight, based on the product to be cleaved.

5. A process as claimed in one of claims 1 to 3, wherein the amount of water added to the product to be cleaved is from 1 to 10% by weight, based on the product to be cleaved.

6. A process as claimed in one of claims 1 to 5, wherein the process is carried out in the presence of molecular oxygen.

7. A process as claimed in one of claims 1 to 6, wherein the product to be cleaved is cleaved at from 150° C. to 250° C.

8. A process as claimed in one of claims 1 to 7, wherein the acid added to the product to be cleaved is a mineral acid and/or an organic acid different from monomeric and oligomeric (meth)acrylic acid.

9. A process as claimed in claim 8, wherein the amount of added acid different from monomeric and oligomeric (meth)acrylic acid is from 1 to 20% by weight, based on the product to be cleaved.

10. A process as claimed in claim 8, wherein the amount of added acid different from monomeric and oligomeric (meth)acrylic acid is from 5 to 15% by weight, based on the product to be cleaved.

11. A process as claimed in one of claims 1 to 10, wherein the cleavage is carried out at reduced pressure (<1 atm).

12. A process as claimed in one of claims 1 to 11, wherein a stripping gas is conducted through the product to be cleaved to remove the cleavage products.

13. A process as claimed in claim 12, wherein the stripping gas used is an oxygen-containing gas.

14. A process as claimed in one of claims 1 to 13, wherein the resulting cleavage products are recycled directly to the esterification.

15. A process as claimed in one of claims 1 to 14, wherein the alkanol is a C₁-C₈-alkanol.

16. A process as claimed in claim 15, wherein the alkanol is n-butanol or 2-ethylhexanol.
